(19) United States
(12) Patent Application Publication
(10) Pub. No.: US 2014/0343117 A1
(43) Pub. Date: Nov. 20, 2014

(54) METHODS AND PRODUCTS TO DIAGNOSE AND TREAT HEATINESS

(71) Applicants: David Lungpao Chang, Las Vegas, NV (US); Zheng Yang, Guangzhou (CN); ZhaoSheng Huang, Guangzhou (CN); SiJun Liu, Guangzhou (CN)

(72) Inventors: David Lungpao Chang, Las Vegas, NV (US); Zheng Yang, Guangzhou (CN); ZhaoSheng Huang, Guangzhou (CN); SiJun Liu, Guangzhou (CN)

(73) Assignee: COLGATE-PALMOLIVE COMPANY, New York, NY (US)

(21) Appl. No.: 14/365,862

(22) PCT Filed: Dec. 21, 2011
(86) PCT No.: PCT/CN2011/002149

§ 371 Date: Jun. 16, 2014

(51) Int. Cl. A61K 31/09; A61K 31/365

(52) U.S. Cl. CPC A61K 31/09; A61K 31/365
USPC 514/425

(57) ABSTRACT

Described herein are methods of diagnosing and/or classifying heatiness, a syndrome recognized in traditional Chinese Medicine, comprising assessing a patient by checking for selected symptoms, and assigning a score to the patient based on the symptoms exhibited. The invention further provides methods of treatment and the use of oral care compositions to treat heatiness.

### BACKGROUND

“Heatiness” is a term used to describe symptoms associated with excessive “internal heat” in our body, which form a syndrome recognized in Traditional Chinese Medicine (TCM). Heatiness is characterized by dryness of mouth, redness, swelling, heat, and pain. Different types of heatiness are recognized, for example, sthenia fire, e.g., characterized by red face/eyes/tongue, and rapid strong pulse, and deficient fire, e.g., sleepless, dry mouth, nosebleed, and rapid weak pulse. Fundamentally, heatiness is considered to be a maladjustment of the balance of yin-yang required to maintain health. The goal of medication under TCM principles is to adjust the body's balance and restore health.

Under TCM principles, heatiness is viewed as the clinical manifestation of exogenous evils transforming into heat or internal depression turning into fire. It exhibits somewhat different symptoms in different parts of the body. The usual symptoms of heatiness within the mouth for example are as follows: boils of tongue and lips, bitter taste in mouth and bad breath, dry mouth and cheilosis, orolingual sores, swelling and reddish of gingiva, toothache and bleeding gum, red tongue with yellowish furry coating, reddish margin of tongue without furry coating. These local symptoms overlap with symptoms of diseases such as gingivitis, periodontitis, acute coccus infective stomatitis, herpetic stomatitis, lingual papillitis, acute parotitis and so on, which are described in Western medicine, but under TCM teachings, the mind and body are viewed as an organic whole, so that the local symptoms in the mouth cannot be effectively disassociated and treated separately from the underlying systemic imbalance resulting in heatiness. A simple one-to-one correlation between diseases recognized by Western medicine and heatiness conditions is thus very difficult. The categorization of disease states is different from Western medicine, and treatment of heatiness typically involves particular diets and/or use of traditional Chinese herbal medicines.

There is a long history of developing understanding and treatments for heatiness, and despite poor acceptance and understanding of TCM principles in Western medicine, millions can testify as to the efficacy of TCM approaches. Existing TCM approaches, including heatiness diagnosis, are based largely upon the accumulation of personal experience, subjective opinion, and anecdotal reports. There is a lack of symptomalogical observation and semi-quantitative or quantitative diagnostic standards that would allow a more rigorous and consistent study of heatiness and its treatment, and also allow a better synthesis and understanding of the relationship between diseases as understood and treated under Western medical principles, and heatiness as understood and treated under TCM principles.

### SUMMARY

A clinical study, including patients diagnosed using TCM principles as suffering from heatiness and normal patients, is conducted to document symptoms of heatiness systematically and objectively. The project team semi-randomly selects three areas from south to north, Guangzhou, Hunan and Henan, and collects data as to symptoms familiar in Western medicine and diagnosis using TCM in four examinations of about 120 heatiness cases, 60 healthy cases, and 60 self-control cases in each area. By employment of the Chi-square test, frequency table method and maximum likelihood discriminant method, a gold standard for the diagnosis of fire-heat syndrome is established after statistical analysis. Then by introduction of the method of percentiles, grading of severity of heatiness is designed.

Based on this study, a scoring system is developed, giving different symptoms different weight, and a critical diagnostic score is identified as corresponding to patients suffering from heatiness. In one embodiment, therefore, the invention provides a new method to diagnose whether people have heatiness utilizing a newly established Diagnostic Score Table (DST). The purpose of this invention is to assist the TCM doctor to diagnose heatiness using a semi-quantitative method that utilizes the DST.

In another embodiment, the invention provides a computer program to classify heatiness and aid in heatiness diagnosis, including diagnosis of the particular type of heatiness.

In another embodiment, the invention provides a method of treating heatiness in patients, particularly the oral symptoms of heatiness, comprising administering an oral care product, e.g., a toothpaste or mouthrinse, comprising cooling agents, particularly herbs from TCM.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

In a first embodiment, the invention provides a method (Method 1) of diagnosing heatiness comprising assessing a patient by checking for symptoms comprising at least 20, for example all 27 of the following symptoms listed in Table A, and giving a score to the patient based on the symptoms exhibited.

TABLE A

| Symptoms | value | Symptoms | value | Symptoms | value |
|---|---|---|---|---|---|
| Mouth ulcer | ***23 | Ocular itching | *8 | Low-grade fever | **15 |
| Dry mouth | **14 | Secretion of | **18 | Insomnia | *8 |
| | | the eye | | | |
| Bitter taste | **15 | Tinnitus | **11 | Tantrum | **17 |
| of mouth | | (ear ringing) | | | |
| Halitosis | **17 | Dry pharynx | **13 | Yellow urine | *9 |
| Gum swelling, | **17 | Sore throat | **15 | Constipation | *8 |
| pain or bleeding | | | | | |
| Red tongue | *9 | Acne | *6 | Stuffiness | *6 |
| Dryness in the | **15 | Dizziness | *6 | Yellow musci | **15 |
| nasal cavity | | | | | |
| Nose bleeding | *10 | Scurf | *6 | Frequent and | **11 |
| | | (scales, flakes) | | weak pulse | |
| Dryness of | *9 | Dryness-heat | **17 | Frequent and | **13 |
| the eyes | | | | strong pulse | |

For example, the invention provides
1.1. Method 1 wherein the symptoms are weighted based on their relative contribution to the diagnosis of heatiness, and a diagnostic critical value is assigned to identify patients as suffering from heatiness, wherein patients exhibiting a score in excess of the diagnostic critical value are considered to suffer from heatiness.
1.2. Method 1.1 wherein symptoms in Table A are marked * are given a lower weight, those marked ** are given a medium weight, and those marked *** are given a higher weight.
1.3. Method 1.2 wherein the symptoms assessed are given a weight approximately corresponding to or approximately proportional to the numerical values listed in Table A.
1.4. Method 1.3 wherein the diagnostic critical value for a diagnosis of heatiness is a value of 60 or higher, e.g., at least 63, using the numerical values in Table A, or is a value proportional to or corresponding to that diagnostic critical value.
1.5. Any of the foregoing methods wherein the degree of heatiness is categorized based on severity, e.g. common, serious, or very serious, e.g., using the numerical values in Table A where common heatiness corresponds to a score of 63---120; serious heatiness corresponds to a score of 121---150; and very serious heatiness corresponds to a score of >150.

In another embodiment, the invention provides a method to classify types of heatiness using a Principal Component Analysis (Prin), for example
2. A method (Method 2) of diagnosing a particular type of heatiness comprising assessing a patient for symptoms as set forth on Table B:

TABLE B

| Prin | Heatiness Type | Primary Heatiness Symptoms |
|---|---|---|
| Prin 1 | Sthenia Fires | bitter taste of mouth, halitosis, nasal obstruction, |
| | | tinnitus, dizzy, scurf, dryness-heat, tantrum, yellow |
| | | urine, yellow musci, frequent and strong pulse |
| Prin 2 | Deficient Fire | dry mouth, halitosis, uloncus, dryness in the nasal |
| | | cavity, sore throat, dizzy, dryness-heat, low-grade |
| | | fever, red tongue, frequent and weak pulse |
| Prin 3 | Heart Fire | mouth ulcer, dryness of eye, sore-throat, |
| | | insomnia, yellow musci |
| Prin 4 | Lung-stomach | dry mouth, uloncus, tinnitus, dry pharynx, acne, |
| | Sthenia | constipation, yellow musci |
| Prin 5 | Hepatic and | uloncus, tinnitus, low-grade fever, tantrum, |
| | renal yin | constipation, red tongue, yellow musci, frequent |
| | deficiency Fire | and weak pulse |
| Prin 6 | Asthenia of | nasal obstruction, acne, red tongue, yellow musci, |
| | pulmonary yin | frequent and weak pulse |

2.1. Method 2 wherein the patient receives a diagnosis of heatiness in accordance with any of Method 1 et seq, then is assessed under Method 2 to determine the type of heatiness.
2.2. Method 2 or 2.1 wherein the symptoms assessed from Table B are weighted in accordance with the methods described above for Method 1, e.g., corresponding to the values set forth in Table A, and the patient is given a value for each principal component (Prin) in Table B, wherein the highest value establishes the type of heatiness affecting the patient.
2.3. Any of Method 2-2.2 wherein the Prin value is calculated using Formula 1 below (paragraph 0052).

In another embodiment, the invention provides a machine readable program and a computer wherein the calculations to determine a weighted score for heatiness and/or type of heatiness in accordance with any of Methods 1, et seq. or Methods 2 et seq. are performed by the machine readable program and the computer based on input regarding the presence or absence of relevant symptoms, e.g., as set forth in Table A and/or B. For example, the invention provides a computer-assisted system for self-diagnosis, wherein a consumer enters data regarding the presence or absence of symptoms as listed in Table A, e.g., via a website, and the data is uploaded into a calculating program, e.g., a spreadsheet program such as Microsoft Excel, to permit calculation of a heatiness diagnostic score, and/or heatiness type or Prin value, e.g. in accordance with Table B, and the relevant value, e.g., score and/or prin value, is then displayed to the patient. Optionally, based on the score, information regarding heatiness and appropriate methods of treatment is also provided to the consumer.

In another embodiment, the invention provides
3. A method of treatment (Method 3) comprising diagnosing a patient in accordance with any of Methods 1 or 2 as suffering from heatiness, and treating the patient by administering an oral care product, e.g., a toothpaste or a mouthwash, comprising an effective amount of an antiheatiness agent.
3.1. Method 3 wherein the antiheatiness agent is recognized in TCM, for example comprising one or more anti-heatiness agents selected from: berberine and jateorhizine from rhizome of Chinese goldthread, baicalin from root of Baikal skullcap, matrine and oxymatrine from root of lightyellow sophora, andrographolide from common andrographis herb, houttuynine sodium bisulfate from herb of heartleaf houttuynia, mangiferin from rhizome of anemarrhena, and alkanin from redroot gromwell; chrysanthemum lactone from flos chrysanthemi indici, chlorogenic acid from honeysuckle flower, tea polyphenols from tea leaf, and magnolol from bark of magnolia.
3.2. Method 3 or 3.1 wherein the antiheatiness agent comprises a synthetic antibacterial or anti-inflammatory agent.
3.3. Method 3.2 wherein the antiheatiness agent is selected from triclosan, zinc citrate, and combinations thereof, e.g., comprising 0.1-1% triclosan and/or 1-3% zinc citrate;
3.4. Any of the foregoing methods wherein the oral care product is administered to the oral cavity of the patient on at least a daily basis for a period of at least one week or until symptoms subside.

In a further embodiment, the invention provides an oral care product, e.g., a toothpaste or a mouthwash, comprising an effective amount of an antiheatiness agent, for use in a method of treating heatiness, for example to treat a patient diagnosed in accordance with any of Method 1 et seq. or Method 2, et seq. e.g.,
wherein the antiheatiness agent is recognized in TCM, for example comprising one or more anti-heatiness agents selected from: berberine and jateorhizine from rhizome of Chinese goldthread, baicalin from root of Baikal skullcap, matrine and oxymatrine from root of lightyellow sophora, andrographolide from common andrographis herb, houttuynine sodium bisulfite from herb of heartleaf houttuynia, mangiferin from rhizome of anemarrhena, and alkanin from redroot gromwell; chrysanthemum lactone from flos chrysanthemi indici, chlorogenic acid from honeysuckle flower, tea polyphenols from tea leaf, and magnolol from bark of magnolia; and/or
wherein the antiheatiness agent comprises a synthetic antibacterial or anti-inflammatory agent, and/or
wherein the antiheatiness agent is selected from triclosan, zinc citrate, and combinations thereof, e.g.
   comprising 0.1-1% triclosan and/or 1-3% zinc citrate;
   optionally further comprising 0.5-2% methyl vinyl ether/maleic anhydride copolymer; and/or
   optionally further comprising
   1-3% % pyrophosphate
   for example wherein the oral care product is selected from
   (i) a dentifrice comprising 0.3% triclosan, 2% methyl vinyl ether/maleic anhydride copolymer and 0.32% sodium fluoride in a silica base and
   (ii) a dentifrice comprising 1.1% sodium monofluorophosphate, 1.5% methyl vinyl ether/maleic anhydride copolymer, 2.44% tetrapotassium pyrophosphate (TKPP), and 2% zinc citrate trihydrate in a silica base.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### EXAMPLES

### Example 1

### Clinical Assessments and Determination of Statistical Correlation Between Particular Symptoms and Heatiness

Heatiness is a syndrome of over-exuberance of yang heat with hyperactivity caused by direct subjection to warm or hot evil qi or loss of balance between the viscera and bowels, yin and yang, qi and blood within the body so as to lead to hyperactive yang qi. The methodology available now permits pattern identification in regard to disease location, pattern identification in regard to disease nature, and pattern identification in regard to objective pathologies and symptoms and to classification of symptoms and syndromes which can provide guidance for clinical medicine and scientific research and which can be readily assessed by Western health providers without extensive training in TCM. There is a need for quantitative diagnostic standards or prospective clinical epidemiological research and study methodology.

This project uses three sets of data of 761 cases of heatiness respectively collected in Guangzhou in Guangdong province, Changsha in Hunan province and Zhengzhou in Henan province. Research of the cases and data with the same experimental and data analysis methodology is carried out with an aim of probing into TCM symptomatological characteristics of heatiness and establishing diagnostic standards for the syndrome. The project is intended to design a set of intuitionistic, scientific diagnostic standards, where each cumulative symptom is given a score value. Thus physicians are able to give objective evaluation of the severity of heatiness. At the same time, the team tries to categorize the heatiness-heat syndrome by means of principal component analysis to provide a scientific theoretic foundation for treatment of it by pattern identification, and further provide a set of diagnostic and therapeutic standards for research and development of drugs, personal care products and wellness products in connection with heatiness.

Establishment of Diagnosis Standards for Heatiness: Inclusion & Exclusion Standards of Heatiness. Diagnosis standards are in agreement with the part of Symptomology, the State Standardized TCM Clinical Diagnostic Terminology of the PRC (GB/T 16751.2-1997), as follows:
1. Sthenia Fire (Heat) Syndrome: internal exuberant fire generally featured by fever, constant thirst, linking for cold beverage, burning heat in the chest and abdomen, reddening of the face and eyes, bound stools, short voiding of yellow urine, red tongue with dry yellow coating, and rapid or rapid flooding pulse.
2. Asthenia Heat Syndrome: deficiency of yin fluid and internal generation of asthenia heat generally featured by persistent low fever, night sweating, rubeosis of the cheeks appearing in the afternoon, dryness of the mouth and constant desire for water, short voiding of yellow urine, dry bound stools, red tongue with little saliva, and thin rapid pulse.
3. Asthenia Fire Syndrome: deficiency of yin fluids and asthenia fire exuberance and hyperactivity generally featured by vexation and sleeplessness, dryness of the mouth and throat, night sweating, spermatorrhea, rubeosis of the cheeks appearing in the afternoon, short voiding of yellow urine, dry bound stools, and possibly hemoptysis, nosebleed or possibly lingual and oral ulceration with red tongue, shortage of saliva, and thin rapid pulse.

Inclusion Standards for Heatiness: A case can be included when the above symptoms are detected and three members of the team arrive at the same diagnosis separately and independently. Exclusion Standards for Heatiness: Excluded are cases of cancer; cases of infectious diseases such as hepatitis and tuberculosis; cases of mental disease; cases of pregnancy or lactation; and cases diagnosed as non-fire-heat syndrome by three members of the team separately and independently.

Designing the Questionnaire on the Signs and Symptoms: By searching the Wanfang database, China journal Net, VIP resource system, and medical academic papers database of wanfang, 20 keywords are identified corresponding to fire, fire-heat, sthenia fire, asthenia fire, asthenic yin causing excessive pyrexia, yangming meridians sthenia fire, hyperpyrexia of liver, stomach-fire, pulmonary fire, cholic fire, and excessive heart fire. In reference to the description of the clinical manifestations of heatiness in the Differentiation of Febrile Diseases, Cold Damage, New Medical Discoveries, Heart Treatment Methods of Dan Xi, Heart Enlightenment on Medicine, TCM Diagnostics by Zhu Wenfeng, and TCM Diagnostics by Signs & Symptoms by Yao Naili, the Fire-heat Syndromes are listed in order of appearing frequency in literature, and the first 30 signs and symptoms are determined as the indices for the four examinations. Then the questionnaire on the Fire-heat Syndrome is designed with a dichotomization variation (Yes/No).

Questionnaire on the Signs & Symptoms: The questionnaire reads as follows:
This survey is conducted by heatiness Research Team, Faculty of Medicinal Herbs, Guangzhou TCM University, intended to establish the quantitative diagnostic standards for heatiness. You are kindly requested to fill in the questionnaire, of which the tongue part will be finished by the clinical surveyor. The survey data are kept in strict privacy and the team will do nothing but analyze the totality of the data to be obtained. If you agree to participate in the survey and give your answers according to facts, you are making a great contribution to the mission of establishing TCM quantitative symptomatology and we will extend thanks to you for your cooperation).

Number:

| 1. | Basic Data: | | | |
|---|---|---|---|---|
| Name: | Gender: | Age: | Occupation: | Birth Place: |

Address:

| | | |
|---|---|---|
| Tel.: | E-mail: | |
| 2. | Do you have oral ulceration? | No (0) Yes (1) |
| 3. | Are you constant thirsty? | No (0) Yes (1) |
| 4. | Do you have bitter taste? | No (0) Yes (1) |
| 5. | Do you have halitosis? | No (0) Yes (1) |
| 6. | Are you constantly hungry? | No (0) Yes (1) |
| 7. | Do you have swollen gums or gum bleed? | No (0) Yes (1) |
| 8. | Is your nose stuffy? | No (0) Yes (1) |
| 9. | Do you feel your nose dry? | No (0) Yes (1) |
| 10. | Do you have nosebleed? | No (0) Yes (1) |
| 11. | Do you feel your eyes dry? | No (0) Yes (1) |
| 12. | Do you feel your eyes itchy? | No (0) Yes (1) |
| 13. | Are your eyes red? | No (0) Yes (1) |
| 14. | Di you feel recently gum in your eyes? | No (0) Yes (1) |
| 15. | Do you feel tinnitus often or just recently? | No (0) Yes (1) |
| 16. | Do you feel your throat dry? | No (0) Yes (1) |
| 17. | Do you have sore swollen throat? | No (0) Yes (1) |
| 18. | Do you cough? | No (0) Yes (1) |
| 19. | Do you have acne on the face? | No (0) Yes (1) |
| 20. | Do you have vertigo? | No (0) Yes (1) |
| 21. | Do you have much scurf? | No (0) Yes (1) |
| 22. | Do you feel vexing hot? | No (0) Yes (1) |
| 23. | Do you have low fever? | No (0) Yes (1) |
| 24. | Do you have insomnia and dreaminess? | No (0) Yes (1) |
| 25. | Do you suffer from irascibility? | No (0) Yes (1) |
| 26. | Is your urine yellow scanty? | No (0) Yes (1) |
| 27. | Are your stools dry bound? | No (0) Yes (1) |
| 28. | Is your tongue reddened? | No (0) Yes (1) |
| 29. | Is the tongue coating yellow? | No (0) Yes (1) |
| 30. | Is your pulse thin and rapid? | No (0) Yes (1) |
| 31. | is your pulse flooding and rapid? | No (0) Yes (1) |

| | |
|---|---|
| Data Collector: | Collection Location: |

| | | | | | |
|---|---|---|---|---|---|
| Collection Time: | yr | mon | day | hour | minute |

Diagnostic Results of Surveyor:

Sample size: In this study, there are 30 items to be studied. In most cases, the sampling content is preferably no less than five times the item number. In this study, accordingly, at least 180 cases are chosen in each area. The 180 cases of the study group and 60 cases of the self-control group undergo preliminary biomarker assessments to confirm that they met the statistic requirements for the sampling content in the one-factor analysis of variance, paired difference test, Chi-square test, relevance test, and principal component analysis. Through semi-randomized sampling, three areas of different latitudes in China are selected: Guangzhou, Hunan and Henan. Because the survey was carried out in the metropolitan areas with enormous populations, where complete randomized sampling was impossible, semi-randomized sampling is adopted.

Statistical Methods: Through the Chi-square test, diagnostic parameters are excluded where the fire-heat cases and the healthy cases showed no difference; by means of conditional probability conversion method, the score value table of the pattern signs and symptoms is designed; by the maximum likelihood discriminant method, the quantitative diagnostic threshold values are set. According to the test and research methods of clinical epidemiological diagnosis, retrospective and prospective inspections of the quantitative diagnostic standards are conducted.

Establishment of Heatiness Symptom Indices: The clinical data of 121 heatiness cases and 60 healthy cases collected in Guangzhou are examined. After the Chi-square test, those symptoms which showed differences P>0.05 in the four examinations are excluded. Those symptoms that had statistical significance are retained.

TABLE 1
Chi-Square Test Table of Signs & Symptoms of the Heaty and Healthy Cases

| | Healthy | Healthy | Heaty | Heaty | Chi-Square | |
|---|---|---|---|---|---|---|
| Symptoms | Negative | Positive | Negative | Positive | Value | P |
| Ulceration | 60 | 0 | 39 | 82 | 74.340 | 0.000 |
| Oral dryness | 50 | 10 | 20 | 101 | 75.475 | 0.000 |
| Bitter taste | 59 | 1 | 84 | 37 | 20.214 | 0.000 |
| Halitosis | 60 | 0 | 81 | 40 | 25.462 | 0.000 |
| Constant hungering | 45 | 15 | 87 | 34 | 0.195 | 0.659 |
| Swollen gums with | 55 | 5 | 23 | 98 | 86.349 | 0.000 |
| pain or bleeding | | | | | | |
| Stuffy nose | 55 | 5 | 89 | 32 | 8.092 | 0.004 |
| Nasal dryness | 58 | 2 | 57 | 64 | 42.523 | 0.000 |
| Nosebleed | 60 | 0 | 109 | 12 | 6.373 | 0.012 |
| Ocular dryness | 50 | 10 | 46 | 75 | 33.071 | 0.000 |
| Ocular itch | 57 | 3 | 87 | 34 | 13.160 | 0.000 |
| Red eyes | 59 | 1 | 113 | 8 | 2.076 | 0.150 |
| Eye gum | 59 | 1 | 66 | 55 | 35.994 | 0.000 |
| Tinnitus | 59 | 1 | 101 | 20 | 20.984 | 0.000 |
| Throat dryness | 57 | 3 | 56 | 65 | 40.591 | 0.000 |
| Sore throat | 59 | 1 | 83 | 38 | 20.984 | 0.000 |
| Sputum | 57 | 3 | 106 | 15 | 2.450 | 0.117 |
| Acne | 48 | 12 | 55 | 66 | 19.519 | 0.000 |
| Vertigo | 55 | 5 | 90 | 31 | 7.522 | 0.006 |
| Scurf | 55 | 5 | 89 | 32 | 8.092 | 0.004 |
| Vexing heat | 60 | 0 | 83 | 38 | 23.85 | 0.000 |
| Low fever | 60 | 0 | 89 | 32 | 19.276 | 0.000 |
| Insomnia | 53 | 7 | 57 | 64 | 28.596 | 0.000 |
| Irascibility | 59 | 1 | 67 | 54 | 34.998 | 0.000 |
| Yellow urine | 55 | 5 | 72 | 49 | 19.821 | 0.000 |
| Constipation | 53 | 7 | 61 | 60 | 24.739 | 0.000 |
| Reddened tongue | 58 | 2 | 97 | 24 | 8.879 | 0.003 |
| Yellow tongue | 59 | 1 | 83 | 38 | 20.984 | 0.000 |
| coating | | | | | | |
| Thin rapid pulse | 55 | 5 | 62 | 59 | 28.681 | 0.000 |
| Rapid flooding | 60 | 0 | 104 | 17 | 9.304 | 0.002 |
| pulse | | | | | | |

Design of the Diagnostic Score Table:

The data collected through the four examinations underwent the maximum likelihood analysis and then listed to get the conditional probability P. The symptoms of constant hungering, red eyes and sputum are P>0.05, showing that they did not differ significantly between the healthy and the heatiness cases. They are, therefore, excluded going forward. By employing the frequency table, symptom occurrence frequency which is called conditional probability P is brought into an index table after being calculated and categorized.

TABLE 2
Frequency Table

| Symptom | Index | Symptom | Index | Symptom | Index |
|---|---|---|---|---|---|
| occurrence % | Value | occurrence % | Value | occurrence % | Value |
| 0-- | −10 | 4.5-- | −3 | 23.0-- | 4 |
| 1.1-- | −9 | 6.0-- | −2 | 29.0-- | 5 |
| 1.4-- | −8 | 7.0-- | −1 | 36.0-- | 6 |
| 1.8-- | −7 | 9.0-- | 0 | 45.0-- | 7 |
| 2.2-- | −6 | 11.0-- | 1 | 57.0-- | 8 |
| 2.8-- | −5 | 14.0-- | 2 | 71.0-- | 9 |
| 3.5-- | −4 | 18.0-- | 3 | 89.0-- | 10 |

The index values of the healthy cases and the heatiness are respectively deducted by the index values to have two sets of absolute values. The two sets are put together to have the score values of the symptoms

TABLE 3
Index Value of Heatiness Symptoms

| | Healthy | | | | | | Heaty | | | | | | |
|---|---|---|---|---|---|---|---|---|---|---|---|---|---|
| | | | Index | | | Index | | | Index | | | Index | |
| Symptoms | Negative | P(%) | value | Positive | P(%) | value | Negative | P(%) | value | Positive | P(%) | value | Value |
| Ulceration | 60 | 100 | 10 | 0 | 0 | −10 | 39 | 32.2 | 5 | 82 | 67.8 | 8 | 23 |
| Oral dryness | 50 | 83.3 | 9 | 10 | 16.7 | 2 | 20 | 16.5 | 2 | 101 | 83.5 | 9 | 14 |
| Bitter taste | 59 | 98.3 | 10 | 1 | 1.7 | −8 | 84 | 69.4 | 8 | 37 | 30.6 | 5 | 15 |
| Halitosis | 60 | 100 | 10 | 0 | 0 | −10 | 81 | 66.9 | 8 | 40 | 33.1 | 5 | 17 |
| Swollen | 55 | 91.7 | 10 | 5 | 8.3 | −1 | 23 | 19 | 3 | 98 | 81 | 9 | 17 |
| gums with | | | | | | | | | | | | | |
| pain or | | | | | | | | | | | | | |
| bleed | | | | | | | | | | | | | |
| Stuffy nose | 55 | 91.7 | 10 | 5 | 8.3 | −1 | 89 | 73.6 | 9 | 32 | 26.4 | 4 | 6 |
| Nasal | 58 | 96.7 | 10 | 2 | 3.3 | −5 | 57 | 47.1 | 7 | 64 | 52.9 | 7 | 15 |
| dryness | | | | | | | | | | | | | |
| Nosebleed | 60 | 100 | 10 | 0 | 0 | −10 | 109 | 90.1 | 10 | 12 | 9.9 | 0 | 10 |
| Ocular | 50 | 83.3 | 9 | 10 | 16.7 | 2 | 46 | 38 | 6 | 75 | 62 | 8 | 9 |
| dryness | | | | | | | | | | | | | |
| Ocular itch | 57 | 95 | 10 | 3 | 5 | −3 | 87 | 71.9 | 9 | 34 | 28.1 | 4 | 8 |
| Ocular gum | 59 | 98.3 | 10 | 1 | 1.7 | −8 | 66 | 54.5 | 7 | 55 | 45.5 | 7 | 18 |
| Tinnitus | 59 | 98.3 | 10 | 1 | 1.7 | −8 | 101 | 83.5 | 9 | 20 | 16.5 | 2 | 11 |
| Throat | 57 | 95 | 10 | 3 | 5 | −3 | 56 | 46.3 | 7 | 65 | 53.7 | 7 | 13 |
| dryness | | | | | | | | | | | | | |
| Sore throat | 59 | 98.3 | 10 | 1 | 1.7 | −8 | 83 | 68.6 | 8 | 38 | 31.4 | 5 | 15 |
| Acne | 48 | 80 | 9 | 12 | 20 | 3 | 55 | 45.5 | 7 | 66 | 54.5 | 7 | 6 |
| Vertigo | 55 | 91.7 | 10 | 5 | 8.3 | −1 | 90 | 74.4 | 9 | 31 | 25.6 | 4 | 6 |
| Scurf | 55 | 91.7 | 10 | 5 | 8.3 | −1 | 89 | 73.6 | 9 | 32 | 26.4 | 4 | 6 |
| Vexing heat | 60 | 100 | 10 | 0 | 0 | −10 | 83 | 68.6 | 8 | 38 | 31.4 | 5 | 17 |
| Low fever | 60 | 100 | 10 | 0 | 0 | −10 | 89 | 73.6 | 9 | 32 | 26.4 | 4 | 15 |
| Insomnia | 53 | 88.3 | 9 | 7 | 11.7 | 1 | 57 | 47.1 | 7 | 64 | 52.9 | 7 | 8 |
| Irascibility | 59 | 98.3 | 10 | 1 | 1.7 | −8 | 67 | 55.4 | 7 | 54 | 44.6 | 6 | 17 |
| Yellow | 55 | 91.7 | 10 | 5 | 8.3 | −1 | 72 | 59.5 | 8 | 49 | 40.5 | 6 | 9 |
| urine | | | | | | | | | | | | | |
| Constipation | 53 | 88.3 | 9 | 7 | 11.7 | 1 | 61 | 50.4 | 7 | 60 | 49.6 | 7 | 8 |
| Reddened | 58 | 96.7 | 10 | 2 | 3.3 | −5 | 97 | 80.2 | 9 | 24 | 19.8 | 3 | 9 |
| tongue | | | | | | | | | | | | | |
| Yellow | 59 | 98.3 | 10 | 1 | 1.7 | −8 | 83 | 68.6 | 8 | 38 | 31.4 | 5 | 15 |
| tongue | | | | | | | | | | | | | |
| coating | | | | | | | | | | | | | |
| Thin rapid | 55 | 91.7 | 10 | 5 | 8.3 | −1 | 62 | 51.2 | 7 | 59 | 48.8 | 7 | 11 |
| pulse | | | | | | | | | | | | | |
| Rapid | 60 | 100 | 10 | 0 | 0 | −10 | 104 | 86 | 9 | 17 | 14 | 2 | 13 |
| flooding | | | | | | | | | | | | | |
| pulse | | | | | | | | | | | | | |

Calculating the Diagnostic Threshold Values:

The maximum likelihood discriminant method is introduced to calculate the threshold values. This method is in common use in TCM quantitative symptomological diagnosis. Based on the probability rate, the diagnostic indices for a category of disease are derived from statistic calculation of a large amount of collected cases. These indices are related with the clinical manifestations of a particular case and then the diagnostic indices are cumulated to get the algebraic sum. Suppose all indices in the diagnosis table are “−” at the outset. Totaling of 27 “−” indices in Table 3, the index sum of the fire-heat syndrome is 202. In the same way we get the index sum of the non fire-heat syndrome, which is 265. Now the relative index sum of the fire-heat syndrome is 63. This implies that only when the “+” index sum is above 63 can a fire-heat case be diagnosed. 63, therefore is the diagnostic threshold value for heatiness.

Usage of the Quantitative Diagnosis Table:

When the symptomological score values of a case are totaled, if the sum is above the threshold value, a fire-heat case can be diagnosed. The higher the sum, the more severe the case. This can also be used as a criterion for comparison of the syndromes of one and the same case at different times.

### Diagnostic Score Tables Derived in the Three Areas

TABLE 4
Guangzhou Diagnostic Score Table

| Symptoms | Value | Symptoms | Value | Symptoms | Value |
|---|---|---|---|---|---|
| Ulceration | 23 | Ocular itch | 8 | Low fever | 15 |
| Oral dryness | 14 | Eye gum | 18 | Insomnia | 8 |
| Bitter taste | 15 | Tinnitus | 11 | Irascibility | 17 |
| Halitosis | 17 | Dry throat | 13 | Yellow urine | 9 |
| Swollen gum | 17 | Sore throat | 15 | Constipation | 8 |
| with pain or | | | | | |
| bleeding | | | | | |
| Stuffy nose | 6 | Acne | 6 | Reddened | 9 |
| | | | | tongue | |
| Nasal dryness | 15 | Vertigo | 6 | Yellow tongue | 15 |
| | | | | coating | |
| Nosebleed | 10 | Scurf | 6 | Thin rapid | 11 |
| | | | | pulse | |
| Ocular dryness | 9 | Vexing heat | 17 | Rapid flooding | 13 |
| | | | | pulse | |

The diagnostic threshold value is 63; if the total of diagnostic score is more than 63, it can be concluded that a person has heatiness.

Diagnostic Score Table Derived from the Data Collected in Henan: The inclusion standards and data collection methods are the same as in Guangzhou

TABLE 5
Henan Diagnostic Score Table

| Symptoms | Value | Symptoms | Value | Symptoms | Value |
|---|---|---|---|---|---|
| Ulceration | 21 | Ocular itch | 17 | Low fever | 9 |
| Oral dryness | 14 | Eye gum | 14 | Insomnia | 12 |
| Bitter taste | 16 | Tinnitus | 14 | Irascibility | 22 |
| Halitosis | 18 | Throat dryness | 15 | Yellow urine | 14 |
| Swollen gums with | 13 | Sore throat | 21 | Constipation | 12 |
| pain or bleeding | | | | | |
| Stuffy nose | 12 | Acne | 10 | Reddened | 18 |
| | | | | tongue | |
| Nasal dryness | 13 | Vertigo | 19 | Yellow tongue | 20 |
| | | | | coating | |
| Nosebleed | 14 | Scurf | 8 | Thin rapid | 15 |
| | | | | pulse | |
| Ocular dryness | 10 | Vexing heat | 12 | Rapid flooding | 15 |
| | | | | pulse | |

The diagnostic threshold value is 84.

A Diagnostic Score Table is derived from synthesized data in Guangzhou and Henan

TABLE 6
Diagnostic Score Table Derived from Synthesized Data in Guangzhou
and Henan

| Symptoms | Value | Symptoms | Value | Symptoms | Value |
|---|---|---|---|---|---|
| Ulceration | 22 | Ocular itch | 12 | Low fever | 8 |
| Oral dryness | 14 | Eye gum | 15 | Insomnia | 9 |
| Bitter taste | 15 | Tinnitus | 13 | Irascibility | 20 |
| Halitosis | 17 | Throat dryness | 13 | Yellow urine | 12 |
| Swollen gums with | 15 | Sore throat | 19 | Constipation | 11 |
| pain or bleeding | | | | | |
| Stuffy nose | 10 | Acne | 9 | Reddened | 15 |
| | | | | tongue | |
| Nasal dryness | 14 | Vertigo | 12 | Yellow tongue | 18 |
| | | | | coating | |
| Nosebleed | 14 | Scurf | 7 | Thin rapid | 11 |
| | | | | pulse | |
| Ocular dryness | 10 | Vexing heat | 18 | Rapid flooding | 14 |
| | | | | pulse | |

The diagnostic threshold value is 74.

A Diagnostic Score Table is derived from the data collected in Hunan

TABLE 7
Hunan Diagnostic Score Table

| Symptoms | Value | Symptoms | Value | Symptoms | Value |
|---|---|---|---|---|---|
| Ulceration | 13 | Ocular itch | 4 | Low fever | 1 |
| Oral dryness | 7 | Eye gum | 7 | Insomnia | 8 |
| Bitter taste | 7 | Tinnitus | 7 | Irascibility | 6 |
| Halitosis | 3 | Throat dryness | 7 | Yellow urine | 10 |
| Swollen gums with | 8 | Sore throat | 12 | Constipation | 7 |
| pain or bleeding | | | | | |
| Stuffy nose | 10 | Acne | 5 | Reddened | 9 |
| | | | | tongue | |
| Nasal dryness | 4 | Vertigo | 10 | Yellow tongue | 13 |
| | | | | coating | |
| Nosebleed | 4 | Scurf | 3 | Thin rapid | 9 |
| | | | | pulse | |
| Ocular dryness | 2 | Vexing heat | 9 | Rapid flooding | 15 |
| | | | | pulse | |

The diagnostic threshold value is 43.

Data collected in the three areas are synthesized, by the above mentioned method, and the maximum likelihood method yields a synthesized Diagnostic Score table.

TABLE 8
Diagnostic Score Table Derived from Synthesized Data in the Three Areas

| Symptoms | Value | Symptoms | Value | Symptoms | Value |
|---|---|---|---|---|---|
| Ulceration | 20 | Ocular itch | 9 | Low fever | 8 |
| Oral dryness | 11 | Eye gum | 12 | Insomnia | 10 |
| Bitter taste | 13 | Tinnitus | 9 | Irascibility | 13 |
| Halitosis | 11 | Throat dryness | 12 | Yellow urine | 10 |
| Swollen gums with | 13 | Sore throat | 16 | Constipation | 9 |
| pain or bleeding | | | | | |
| Stuffy nose | 9 | Acne | 7 | Reddened | 12 |
| | | | | tongue | |
| Nasal dryness | 9 | Vertigo | 10 | Yellow tongue | 16 |
| | | | | coating | |
| Nosebleed | 9 | Scurf | 6 | Thin rapid | 10 |
| | | | | pulse | |
| Ocular dryness | 8 | Vexing heat | 11 | Rapid flooding | 15 |
| | | | | pulse | |

The diagnostic threshold value is 66.

Evaluation of the Diagnostic Efficiency by ROC Curve: Sensitivity refers to the ratio of correctly diagnosed disease cases by a screening method while specificity to the ratio of correctly determined cases as healthy by a screening method. They are the two common indices demonstrating the diagnostic trial accuracy. The higher the diagnostic trial sensitivity, the lower the missed diagnostic rate. The higher the specificity, the lower the misdiagnostic rate. The two indices are generally used to evaluate the reliability of a screening method in diagnostic trials.

Drawing a curve with the true positive rate (sensitivity) as the longitudinal coordinate and the false positive rate (1-specificity) as the abscissa, we get a receiver operating characteristic curve (ROC Curve), which is also called the sensitivity curve. The dots on the curve reflect the same sensitivity, and each of them is a response to one and the same signal stimulation. Taking sensitivity as the ordinate axis and specificity as the abscissa axis, the size of the integrated areas under the curve is closely related to the reliability of a diagnostic trial. The ROC curve is composed, based on a series of various binary modes (cut-off value or determination threshold), of countless pairs of true positive rate and false positive rate derived by countless critical values. We appraise a diagnostic efficiency through calculating the area under the ROC curve. The nearer the ROC curve is to the left upper corner, the more accurate the trial is suggested to be. The ROC curve may be used to inspect the performance and characteristics of a trial in a general way, and the results of the inspection are more objective and reliable.

In connection with the data collected in the three areas, all the standards of trial diagnosis centers are homogenous. In spite of the difference in the threshold values between the three areas, the trial diagnosis centers in these areas do not differ in diagnosis results (sensitivity and specificity) when using their own standards. Owing to the difference in sampling, the difference trial diagnosis centers derive different threshold values but this does not affect the diagnosis of heatiness.

Sensitivity & Specificity of the Diagnostic Score Table: Sensitivity
(SE) and specificity (SP) are two indices used to test the accuracy of
diagnostic trial. SE = a/(a + c)*100% and SP = d/(b + d)*100%.

| | | Diagnosis according | |
|---|---|---|---|
| | | to the table | |
| | | Heatiness | Healthy |
| Clinical diagnosis | Heatiness | a | b |
| (by clinicians) | Healthy | c | d |

Note:
a represents the number of heatiness cases diagnosed both by the clinician and according to the table;
b, the number of heatiness cases diagnosed by the clinician but of healthy cases according to the table (score value < threshold value);
c, the number of healthy cases diagnosed by the clinician but of heatiness cases according to the table (score value > threshold value);
d, the number of healthy cases diagnosed both by the clinician and according to the table.

Retrospective inspection is a study of a group of subjects selected during a period of time before the study. The subjects are divided into the study and control groups according to the present knowhow. This method encompasses a period of time from a certain past time till the present day. Guangzhou diagnostic score table was derived from analysis of the data collected in Guangzhou. In order of time when the data of cases were collected, this table is limited to retrospection of this set of cases. The sampling content (N) that Henan table gives retrospection includes the sum of the cases collected in Guangzhou and Henan while the sampling content (N) that Hunan table gives retrospection includes the sum of the cases collected in Guangzhou, Henan and Hunan.

| | Sampling content | | |
|---|---|---|---|
| Diagnostic Score table | (N) | SE | SP |
| Guangzhou table | 121 | 97.50% | 98.30% |
| | 60 | | |
| Henan Table | 264 | 93.90% | 100% |
| | 134 | | |
| Synthesis of Guangzhou | 264 | 96.70% | 100% |
| & Henan | 134 | | |
| Hunan Table | 386 | 98.66% | 91.35% |
| | 195 | | |
| Synthesis of 3 areas | 386 | 98.90% | 88.43% |
| | 195 | | |

Prospective inspection is to select cases and then make a study of them with a predetermined method, followed by follow-up research. Finally evaluation is performed in the schemed time. All cases that are consistent with the original scheme are included (not merely including valid cases) to exhibit all the results of the survey. In this study, in order of time when the cases are collected, the sampling content (N) of the prospective inspection in Guangzhou table (GZ) includes all the cases collected in Henan and Hunan; the sampling content (N) of the prospective inspection in Henan table (HU) includes all the cases collected in Hunan.

| Diagnostic Score table | N | SE | SP |
|---|---|---|---|
| GZ (Henan & Hunan cases) | 265 | 95.85% | 91.85% |
| | 135 | | |
| HU (Hunan cases) | 122 | 94.78% | 80.88% |
| | 61 | | |
| Synthesis of GX & HU | 122 | 94.83% | 82.09% |
| (Hunan cases) | 61 | | |

From the above analysis, it is seen that both the sensitivity and the specificity of either prospective or retrospective inspections based on Guangzhou diagnostic score table are above 90%. In the retrospective inspection, the different criteria of the three areas differ little in terms of sensitivity and specificity, whereas in the prospective inspection, Guangzhou criteria prove to be the highest in sensitivity and specificity. For that reason, we select a Diagnostic Score Table threshold value of 63 as the standard for future studies.

The test of normality of the total of cases collected in Guangzhou, Henan and Hunan in reference to Guangzhou diagnostic score table has shown that the score values of severity of the fire-heat cases are consistent with the normal distribution. Statistically, if a digital index is consistent with the normal distribution, percentage grading is applicable.

According to the method of percentiles (p): P33.3=120.63≈120; P66.7=149.37≈150. Heatiness can be classified into three degrees:
common: 63---120;
serious: 121---150;
very serious: >150

### Example 2

### Categorization of Types of Heatiness Using Computer Method

So far there is little agreement on the categorization of heatiness in the field of TCM. Some argue for five types, hyperactivity of heart fire, liver-gallbladder damp heat, hyperactivity of stomach fire, splenic asthenia with wet heat, and hyperactivity of fire caused by of YIN deficiency, while some divide the pattern into four types, YIN-deficiency, splenic asthenia, lung-stomach with wet heat, Chong-Ren meridian stagnated heat. Without rigorous underlying scientific analysis, such categorizations merely rely on clinical experience. In this study, through principal component analysis and with the statistical method, the pattern is divided into two categories with six major components, sthenia fire exuberance-hyperactivity and asthenia fire up-flaming. In reference to the viscera and bowels, they are sub-divided into heart fire up-flaming, lung-stomach sthenia fire, liver-kidney yin deficiency, and lung yin deficiency. This categorizing system is of clinical instructive significance.

Principal component analysis is a data statistical analysis method used to re-arrange the original variants to become a new set of unrelated synthesized variants, from which a smaller number of synthesized variants are drawn out to reflect the maximal message of the data of the original variants. In research practice, to make an all-round analysis, a great amount of relevant variants (or factors) should be taken into consideration for any relevant variant may contain certain useful message. In the study of an issue of multiple variants with this statistic analysis method, excessive amounts of variants may greatly complicate the study. An ideal option is to have a small amount of variants enough to reflect great quantities of messages. In many cases, some variants are mutually relevant to a certain extent. These variants invariably overlay each other in regard to the messages they convey. The principal component analysis may reduce the number of the original variants to the smallest possible number of variants, none of which are correlated, but which preserve the largest possible amount of original messages.

For the study, 27 symptoms having significant correlation with heatiness are identified and listed in a questionnaire. 121 heatiness cases and 60 healthy cases are collected from volunteers and the in- and out-patients of the First Hospital Affiliated to Guangzhou TCM University, and Guangdong Hospital of TCM (presence of a syndrome symbolized by +, and absence by −). The variants X1, X2 . . . X27 represent respectively the 27 original symptoms such as ulceration, oral dryness . . . rapid flooding pulse. As the sampling content, generally speaking, should be no less than 5 times the number of variants to be studied, and therefore, 181 cases in Guangzhou are selected as the study subjects.

Statistic Methods & Results: In this study the SAS software is used. The heatiness case is input as 1 and the healthy case as 0; presence of a symptom as 1 and absence as 0 for each X among a total of 27 variants. After all these data required are fed, the programming is run and an analysis matrix is obtained in which a total of 27 new variants called Prin covering Prin 1 to Prin 27 are generated. According to the SAS analysis matrix eigenvalue table here lists six equations referring to Prin 1 to 6:

Prin1=−0.126498X1+0.086727X2+0.299185X3+0.273515X4−0.010763X5+0.213240X6+0.152397X7+0.000000X8+0.184174X9−0.297309X10+0.197090X11+0.258311X12+0.014887X13−0.032549X14−0.166092X15+0.303254X16+0.240957X17+0.240633X18−0.035714X19−0.066931X20+0.303381X21+0.297511X22+0.028836X23−0.030138X24+0.216393X25−0.009607X26+0.209046X27

Prin2=−0.086437X1+0.252758X2−0.148801X3+0.211848X4+0.228970X5−0.030979X6+0.322652X7+0.000000X8+0.066846X9+0.071538X10+0.048838X11−0.161170X12+0.186768X13+0.335628X14−0.032686X15+0.254656X16−0.225833X17+0.314932X18+0.248487X19+0.167677X20−0.127144X21+0.034751X22+0.028157X23+0.219598X24−0.105016X25+0.215114X26−0.298328X27

Prin3=0.378719X1+0.143619X2−0.009102X3+0.121777X4−0.193553X5+0.035710X6+0.009849X7+0.000000X8+0.270589X9+0.122303X1 0−0.332240X11+0.107961X12−0.130511X13+0.253006X14+0.037594X15−0.018648X16+0.167569X17+0.197212X18−0.145620X19+0.245516X20+0.072704X21−0.286196X22−0.327930X23+0.156808X24+0.207549X25−0.277828X26−0.033703X27

Prin4=−0.173732X1+0.297523X2−0.049846X3+0.094096X4+0.214588X5+0.066418X6−0.314251X7+0.000000X8+0.009833X9+0.163815X10+0.076741X11+0.313312X12+0.401596X13+0.009704X14+0.372474X15−0.104113X16−0.028338X17+0.022868X18−0.248188X19+0.195279X20−0.141934X21−0.045261X22+0.245389X23−0.123597X24+0.276817X25−0.048356X26+0.033176X27

Prin5=0.114009X1+0.000511X2+0.047872X3−0.228836X4+0.366254X5−0.359098X6−0.065813X7+0.000000X8−0.102602X9−0.064991X10−0.143919X11+0.219212X12−0.227848X13+0.091507X14+0.105051X15−0.005582X16+0.189958X17+−0.030960X18+0.294337X19+0.007216X20+0.230217X21−0.079504X22+0.244101X23+0.347174X24+0.256178X25+0.254279X26+0.128829X27

Prin6=−0.110586X1−0.091813X2−0.185461X3−0.159592X4−0.209874X5+0.337900X6−0.155910X7+0.000000X8+0.196820X9−0.294048X10+0.132200X11−0.191065X12−0.101991X13−0.176167X14+0.351361X15+0.140844X16−0.083134X17+0.065240X18+0.062242X19+0.033147X20−0.242252X21−0.081500X22−0.117893X23+0.406218X24+0.221808X25+0.207765X26+0.142426X27 (Formula 1)

The contribution rates of the six principal components are taken as the weight number to procure the weighted values. Then the synthesized appraisal index of the principal components is derived:

E=0.182Prin1+0.1728Prin2+0.1462Prin3+0.1013Prin4+0.0827Prin5+0.0747Prin6 (Formula 2)

The contribution rates of the new principal components Prin1 to Prin6 are respectively 18.2%, 17.28%, 14.62%, 10.13%, 8.27%, and 7.47%. Judging from this series, it is clear that the 6 principal components embrace 75.98% messages of the original 27 Prin, thus, can be used as main components to represent the original 27 Prin. Each Prin is associated with 27 symptoms represented by X1 to X27 listed in Formula 1. In accordance with the load of Formula 1, those variants whose coefficient absolute values >0.2 are taken out.

In Prin1, the coefficient absolute values of x3 bitter taste, x4 halitosis, x6 stuffy nose, x12 tinnitus, x16 vertigo, x17 scurf, x18, vexing heat, x21 irascibility, x22 yellow urine, x25 yellow urine, and x27 rapid flooding pulse ≧0.2, and therefore, they fell into one category. According to TCM clinical experience, this category is of sthenia fire.

In Prin2, the coefficient absolute values of x2 oral dryness, x4 halitosis, x5 swollen gums with pain or bleeding, x7 nasal dryness, x14 sore throat, x16 vertigo, x18 vexing heat, x19 low fever, x24 reddened tongue, and x26 thin rapid pulse≧0.2. According to TCM clinical experience, this category is of asthenia fire.

In Prin3, the coefficient absolute values of x1 ulceration, x9 ocular dryness, x14 sore throat, x20 insomnia, and x25 yellow tongue coating ≧0.2. According to TCM clinical experience, this category is of heart fire.

In Prin4, the coefficient absolute values of x2 oral dryness, x5 swollen gums with pain or bleeding, x12 tinnitus, x13 throat dryness, x15 acne, x23 constipation and x25 yellow tongue coating≧0.2. According to TCM clinical experience, this category is of lung-stomach sthenia fire.

In Prin5, the coefficient absolute values of x5 swollen gums with pain or bleeding, x12 tinnitus, x19 low fever, x23 constipation, x24 reddened tongue, x25 yellow tongue coating, and x26 thin rapid pulse≧0.2. According to TCM clinical experience, this category is of liver-kidney yin deficiency.

In Prin6, the coefficient absolute values of x6 stuffy nose, x15 acne, x24 reddened tongue, x25 yellow tongue coating, and x26 thin rapid pulse ≧0.2. According to TCM clinical experience, this category is of lung yin deficiency.

By means of principal component analysis, from Henan and Hunan data are derived six TCM-defined types of heatiness, too. The first two principal components cover two major categories, asthenia fire and sthenia fire. In reference to the viscera and bowels, the pattern is divided into heart fire up-flaming, lung-stomach sthenia fire, liver-kidney yin deficiency, and lung yin deficiency.

The 27 defined symptoms are introduced into Formula 1. Presence of a symptom is set as 1 and absence as 0. In a case, for instance, if there is X1 ulceration, the value of X1 is 1, and it is otherwise 0. Based on the formulas derived by principal component analysis, Prin1 to Prin6 is calculated and the absolute values compared. The Prin (a new factor) whose absolute value is the biggest determines heatiness type.

In accordance with this categorization standard, a retrospective inspection is carried out of the data in the three areas. Its diagnosis concordance rate is 71.07% compared with the diagnosis made by the physicians in Guangzhou, and 69.40% compared with that by the physicians in the three areas. This shows that the categorization agrees with the diagnosis of TCM clinicians.

At present, there is no uniform categorizing standard for heatiness. Categorizations available mainly rely on experience, lacking in scientific statistical support. What is more, no large scale survey has ever been conducted. In this study, by introduction of the method of principal component analysis, we classify the pattern into four categories, heart fire up-flaming, lung-stomach sthenia fire, liver-kidney yin deficiency, and lung yin deficiency. The classification agrees basically with the judgment of clinicians. However, as there is not yet a mature uniform judgment standard for heatiness and the diagnosis of the clinicians is often under the sway of some factors such as their experience and knowhow, the diagnosis concordance rate in this study is subject to the influence of the attending physicians. Statistical analysis as described herein allows more objective study and more objective standards for diagnosis, treatment and evaluation of treatment efficacy.

### Example 3

### Treatment of Heatiness Using Toothpaste

Random double blind studies are conducted wherein patients diagnosed with heatiness in accordance with Example 1 are administered different toothpastes:
Three Day Study:
Dentifrice formulation containing 1.1% Sodium Monofluorophosphate, 1.5% PVM/MA Copolymer (Gantrez), 1.3% pyrophosphate, 2% Zinc citrate trihydrate in a silica base (Colgate 360 Whole Mouth Health-Gum Health Toothpaste) vs. Control 1, a commercial toothpaste containing herbs claiming help to relieve gum problems (such as bleeding, gum pain) etc. in silica base with wintergreen flavor)
Seven Day Study:
Dentifrice formulation containing 0.3% Triclosan, 2% PVM/MA Copolymer (Gantrez) and 0.32% sodium fluoride in a silica base (Colgate Total® Triclosan/Gantrez Toothpaste) vs. Dentifrice formulation containing 1.1% Sodium Monofluorophosphate, 1.5% PVM/MA Copolymer (Gantrez), 1.3% pyrophosphate, 2% Zinc citrate trihydrate in a silica base (Colgate 360 Whole Mouth Health-Gum Health Toothpaste) vs. Control 2, a commercial toothpaste containing 1.1% Sodium Monofluorophosphate in a calcium carbonate base with mint flavor”

After 3-day usage, people who use Colgate 360 Whole Mouth Health-Gum Health Toothpaste with 2% Zinc citrate statistically show overall greater reduction in heatiness diagnostic score (DS) reduction compared to those who use control 1. On the 7-day usage, we use LPP Whitening Toothpaste as control. People who use Colgate 360 Whole Mouth Health-Gum Health Toothpaste with 2% Zinc citrate statistically showed overall greater reduction in heatiness diagnostic score, while Colgate Total® Triclosan/Gantrez Toothpaste formula showed even more improvement:

Three day trial (*Diagnostic Score expressed as mean score +/−
standard deviation)

| | | | | Comparison versus | |
|---|---|---|---|---|---|
| | | | | Control 1 | |
| | | Baseline | Three-Day | Percent | |
| TREATMENT | n | DS* | DS* | Difference | Sig. |
| 360 WMH | 30 | 189.2 ± 50.3 | 153.8 ± 48.0 | −10.6% | P < 0.05 |
| Gum Health | | | | | |
| Toothpaste | | | | | |
| Control 1 | 30 | 187.8 ± 37.5 | 172.6 ± 40.9 | | |

Seven day trial (*Diagnostic Score expressed as mean score +/−
standard deviation)

| | | | | Comparison versus | |
|---|---|---|---|---|---|
| | | | | Control 2 | |
| | | Baseline | Seven-Day | Percent | |
| TREATMENT | n | DS* | DS* | Difference | Sig. |
| 360 WMH | 30 | 189.2 ± 50.3 | 115.1 ± 61.6 | −16.2% | P < 0.05 |
| Gum Health | | | | | |
| Toothpaste | | | | | |
| Triclosan/ | 30 | 175.0 ± 45.1 | 97.6 ± 57.1 | −21.3% | P < 0.05 |
| Gantrez | | | | | |
| Toothpaste | | | | | |
| Control 2 | 30 | 178.3 ± 32.1 | 137.4 ± 46.3 | | |

1. A method of diagnosing heatiness comprising assessing a patient by checking for symptoms listed in Table A, and assigning a score to the patient based on the symptoms exhibited
TABLE A

| Symptoms | value | Symptoms | value | Symptoms | value |
|---|---|---|---|---|---|
| Mouth ulcer | ***23 | Ocular itching | *8 | Low-grade fever | **15 |
| Dry mouth | **14 | Secretion of the | **18 | Insomnia | *8 |
| | | eye | | | |
| Bitter taste of | **15 | Tinnitus | **11 | Tantrum | **17 |
| mouth | | (ear ringing) | | | |
| Halitosis | **17 | Dry pharynx | **13 | Yellow urine | *9 |
| Gum swelling, | **17 | Sore throat | **15 | Constipation | *8 |
| pain or bleeding | | | | | |
| Red tongue | *9 | Acne | *6 | Stuffiness | *6 |
| Dryness in the | **15 | Dizziness | *6 | Yellow musci | **15 |
| nasal cavity | | | | | |
| Nose bleeding | *10 | Scurf | *6 | Frequent and | **11 |
| | | (scales, flakes) | | weak pulse | |
| Dryness of | *9 | Dryness-heat | **17 | Frequent and | **13 |
| the eyes | | | | strong pulse | |

2. The method according to claim 1 wherein the symptoms are weighted based on their relative contribution to the diagnosis of heatiness, and a diagnostic critical value is assigned to identify patients as suffering from heatiness, wherein patients exhibiting a score in excess of the diagnostic critical value are considered to suffer from heatiness.

3. The method according to claim 2 wherein symptoms in Table A marked * are given a lower weight, those marked ** are given a medium weight, and those marked *** are given a higher weight.

4. The method according to claim 3 wherein the symptoms assessed are given a weight approximately corresponding to or approximately proportional to the numerical values listed in Table A.

5. The method according to claim 4 wherein the diagnostic critical value for a diagnosis of heatiness is a value of 60 or higher using the numerical values in Table A, or is a value proportional to or corresponding to that diagnostic critical value.

6. The method according to claim 1 wherein the degree of heatiness is categorized based on severity

7. The method according to claim 6 using the numerical values in Table A where common heatiness corresponds to a score of 63---120; serious heatiness corresponds to a score of 121---150; and very serious heatiness corresponds to a score of >150.

8. A method to classify types of heatiness using a Principal Component Analysis (Prin) comprising diagnosing a particular type of heatiness by assessing a patient for symptoms as set forth on Table B:
TABLE B

| Prin | Heatiness Type | Primary Heatiness Symptoms |
|---|---|---|
| Prin 1 | Sthenia Fire | bitter taste of mouth, halitosis, nasal obstruction, |
| | | tinnitus, dizzy, scurf, dryness-heat, tantrum, |
| | | yellow urine, yellow musci, frequent and strong |
| | | pulse |
| Prin 2 | Deficient Fire | dry mouth, halitosis, uloncus, dryness in the nasal |
| | | cavity, sore throat, dizzy, dryness-heat, low-grade |
| | | fever, red tongue, frequent and weak pulse |
| Prin 3 | Heart Fire | mouth ulcer, dryness of eye, sore-throat, |
| | | insomnia, yellow musci |
| Prin 4 | Lung-stomach | dry mouth, uloncus, tinnitus, dry pharynx, acne, |
| | Sthenia | constipation, yellow music |
| Prin 5 | Hepatic and | uloncus, tinnitus, low-grade fever, tantrum, |
| | renal yin | constipation, red tongue, yellow musci, frequent |
| | deficiency | and weak pulse |
| | Fire | |
| Prin 6 | Asthenia of | nasal obstruction, acne, red tongue, yellow musci, |
| | pulmonary yin | frequent and weak pulse |

9. The method of claim 8 wherein the patient receives a diagnosis of heatiness in accordance with any of claims 1-7 then is assessed under the method of claim 8 to diagnose qualitatively the type of heatiness.

10. The method of claim 8 wherein the patient is given a value for each principal component (Prin) in Table B, wherein the Prin with the highest value establishes the type of heatiness affecting the patient.

11. A computer-assisted system for self-diagnosis, wherein a consumer enters data regarding the presence or absence of symptoms as listed in Table A via a website, the data is uploaded into a calculating program to permit calculation of a heatiness diagnostic score and/or heatiness type, and the heatiness diagnostic score and/or heatiness type is then displayed to the patient.

12. The system of claim 11 wherein, based on the heatiness diagnostic score, information regarding heatiness and appropriate methods of treatment is provided to the consumer.

13. A method of treatment comprising diagnosing a patient as suffering from heatiness in accordance with any of the foregoing claims, and treating the patient by administering an oral care product comprising an effective amount of an antiheatiness agent.

14. The method of claim 13 wherein the antiheatiness agent is one or more anti-heatiness agents selected from: berberine and jateorhizine from rhizome of Chinese goldthread, baicalin from root of Baikal skullcap, matrine and oxymatrine from root of lightyellow sophora, andrographolide from common andrographis herb, houttuynine sodium bisulfite from herb of heartleaf houttuynia, mangiferin from rhizome of anemarrhena, and alkanin from redroot gromwell; chrysanthemum lactone from flos chrysanthemi indici, chlorogenic acid from honeysuckle flower, tea polyphenols from tea leaf, and magnolol from bark of magnolia.

15. The method of claim 13 wherein the antiheatiness agent comprises a synthetic antibacterial or anti-inflammatory agent.

16. The method of claim 15 wherein the antiheatiness agent is selected from triclosan, zinc citrate, and combinations thereof.

17. The method of claim 13 wherein the oral care product is a toothpaste.

18. The method of claim 13 wherein the oral care product is administered to the oral cavity of the patient on at least a daily basis for a period of at least one week or until symptoms subside.

19. An oral care product comprising an effective amount of an antiheatiness agent, for use in a method of treating heatiness according to claim 13.

20. The oral care product of claim 19 wherein the antiheatiness agent is one or more anti-heatiness agents selected from: berberine and jateorhizine from rhizome of Chinese goldthread, baicalin from root of Baikal skullcap, matrine and oxymatrine from root of lightyellow sophora, andrographolide from common andrographis herb, houttuynine sodium bisulfite from herb of heartleaf houttuynia, mangiferin from rhizome of anemarrhena, and alkanin from redroot gromwell; chrysanthemum lactone from flos chrysanthemi indici, chlorogenic acid from honeysuckle flower, tea polyphenols from tea leaf, and magnolol from bark of magnolia.

21. The oral care product of claim 19 wherein the antiheatiness agent comprises a synthetic antibacterial or anti-inflammatory agent.

22. The oral care product of claim 21 wherein the antiheatiness agent is selected from triclosan, zinc citrate, and combinations thereof.

23. The oral care product of claim 21 wherein the oral care product is selected from
a dentifrice comprising 0.3% triclosan, 2% methyl vinyl ether/maleic anhydride copolymer and 0.32% sodium fluoride in a silica base and
a dentifrice comprising 1.1% sodium monofluorophosphate, 1.5% methyl vinyl ether/maleic anhydride copolymer, 1.3% pyrophosphate, and 2% zinc citrate trihydrate in a silica base.

24. Use of an oral care product comprising an effective amount of an antiheatiness agent in the manufacture of a medicament for the treatment of heatiness according to claim 13.

25. Use of an oral care product according to any of claims 20 to 23 in the manufacture of a medicament for the treatment of heatiness according to claim 13.
